(19) United States
(12) Patent Application Publication
(10) Pub. No.: US 2023/0076587 A1
(43) Pub. Date: Mar. 9, 2023

(54) ALL-NATURAL HEALING AGENT

(71) Applicant: Shaytee Herbert Abraham Gadson, Hollywood, SC (US)

(72) Inventor: Shaytee Herbert Abraham Gadson, Hollywood, SC (US)

(21) Appl. No.: 17/447,147

(22) Filed: Sep. 8, 2021

(51) Int. Cl. A61K 36/63; A61K 36/53; A61K 33/14; A61K 36/31; A61K 36/899; A61K 36/288; A61K 36/23

(52) U.S. Cl. CPC A61K 36/63; A61K 36/53; A61K 33/14; A61K 36/31; A61K 36/899; A61K 36/288; A61K 36/23

(57) ABSTRACT

A healing composition includes about 80 wt. % olive oil; about 15 wt. % kosher salt; and about 5 wt. % liquid rosemary. The healing composition is made by providing olive oil in a container; adding kosher salt to the container; adding liquid rosemary to the container; and mixing the olive oil, kosher salt, and liquid rosemary.

### BACKGROUND OF THE INVENTION

The present invention relates to an all-natural healing agent and, more particularly, to an olive oil, liquid rosemary, and kosher salt composition.

Currently available all-natural remedies do not have a proven track record with a cross-section of people of various ages, ethnicities, races, creeds, or colors.

As can be seen, there is a need for an effective all-natural remedy.

### SUMMARY OF THE INVENTION

The present invention is a healing remedy for various types of ailments, including chronic pain.

In one aspect of the present invention, a healing composition is provided comprising: about 80 wt. % olive oil; about 4-5 wt. % kosher salt; and about 15 wt. % liquid rosemary.

In another aspect of the present invention, a method of manufacturing a healing composition is provided comprising: providing olive oil in a container; adding kosher salt to the container; adding liquid rosemary to the container; and mixing the olive oil, kosher salt, and liquid rosemary.

These and other features, aspects and advantages of the present invention will become better understood with reference to the following drawings, description, and claims.

### BRIEF DESCRIPTION OF THE DRAWINGS

FIG. 1 is a schematic view of a method of manufacturing a composition according to an embodiment of the present invention; and

FIG. 2 is a flow chart thereof.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a composition comprising olive oil, liquid rosemary, and kosher salt that heals nearly any malady or ailment. Each ingredient has worked for centuries to cure pain and other maladies. In combination, these ingredients provide a potent weapon to fight against pain and various other ailments.

Without being bound by theory, rosemary oil is believed to relieve indigestion, gas, stomach cramping, bloating, and constipation; to stimulate appetite; and to regulate bile production. Rosemary oil is also believed to have anti-inflammatory and pain-relieving properties, to function as an expectorant, and to stimulate scalp circulation. Liquid rosemary is an extract of rosemary oil.

Without being bound by theory, olive oil is known to contain large quantities of oleic acid and antioxidants and is believed to reduce inflammation.

Without being bound by theory, mustard seed contains antioxidants and is believed to reduce inflammation and promote healing of psoriasis.

Kosher salt is preferred in the present composition for its effectiveness.

In some embodiments, the inventive composition comprises olive oil, liquid rosemary, kosher salt, and mustard seed.

In some embodiments, the composition may consist essentially of or may consist of olive oil, liquid rosemary, kosher salt, and mustard seed.

In some embodiments, the inventive composition may further comprise Bermuda Grass.

In some embodiments, the inventive composition may further comprise Dandelion flower.

In some embodiments, the inventive composition may further comprise parsley.

The inventive composition may be manufactured by combining olive oil, liquid rosemary, kosher salt, and mustard seed in a receptacle or bottle. The user may pour in olive oil, add kosher salt, and add liquid rosemary. The user may then mix the ingredients together.

The inventive composition may be applied externally to the body and skin or ingested orally. In some embodiments, the composition is for external use only. The composition is about 80 wt. % olive oil, about 15 wt. % liquid rosemary, and about 4-5 wt. % kosher salt with respect to the portion of the composition consisting of olive oil, rosemary, and kosher salt. Mustard seed may also be present in an amount of up to about 1 wt. %. The composition excludes garlic, lemon zest, and vegetables such as potatoes.

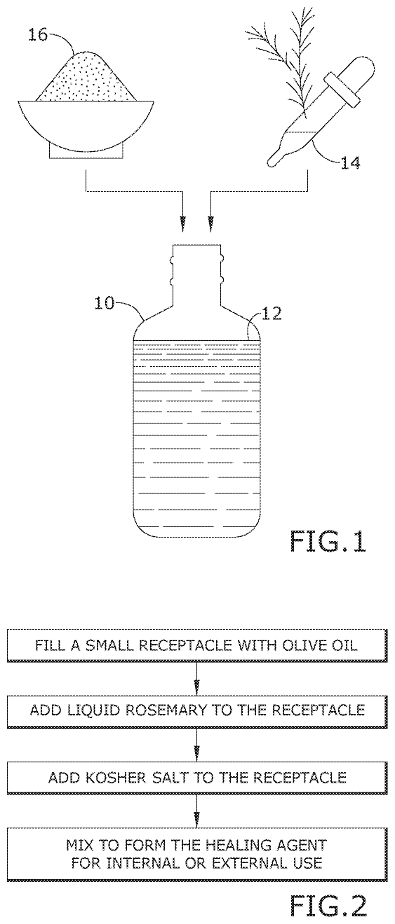

Referring to FIGS. 1 and 2, FIG. 1 illustrates addition of kosher salt 16 and liquid rosemary 14 to a receptacle 10 containing olive oil 12. FIG. 2 provides a flow chart of the steps illustrated in FIG. 1.

It should be understood, of course, that the foregoing relates to exemplary embodiments of the invention and that modifications may be made without departing from the spirit and scope of the invention as set forth in the following claims.

What is claimed is:

1. A healing composition comprising:
a. about 80 wt. % olive oil;
b. about 4 to about 5 wt. % kosher salt; and
c. about 15 wt. % liquid rosemary.

2. The healing composition of claim 1, further comprising a botanical component selected from the group consisting of mustard seed, Bermuda Grass, Dandelion flower, parsley, and combinations thereof.

3. The healing composition of claim 1, formulated for application on skin.

4. The healing composition of claim 1, formulated for oral ingestion.

5. A method of manufacturing a healing composition comprising:
a. providing olive oil in a container;
b. adding kosher salt to the container;
c. adding liquid rosemary to the container; and
d. mixing the olive oil, kosher salt, and liquid rosemary.
